(19) United States
(12) Patent Application Publication
(10) Pub. No.: US 2007/0105927 A1
(43) Pub. Date: May 10, 2007

(54) Amorphous rizatriptan benzoate

(75) Inventors: Ramasubramanian Sridharan, Koparkhairane (IN); Samir Jaivant Naik, Ghantalti Path (IN); Bhupesh Vishnu Aher, Jogeshwari (IN); Nitin Sharad Chandra Pradhan, Manpada (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/584,284

(22) Filed: Oct. 20, 2006

Related U.S. Application Data
(60) Provisional application No. 60/734,561, filed on Nov. 8, 2005.

(30) Foreign Application Priority Data
Oct. 20, 2005 (IN) 1323/MUM/2005

(51) Int. Cl. A61K 31/4196; C07D 403/02

(52) U.S. Cl. 514/383; 548/266.4

(57) ABSTRACT

Rizatriptan benzoate in an amorphous form is disclosed. Also disclosed is a process for preparing rizatriptan benzoate substantially in amorphous form comprising the steps of (a) preparing a solvent solution comprising non-amorphous rizatriptan benzoate and one or more solvents capable of dissolving the non-amorphous rizatriptan benzoate; and (b) recovering the amorphous form of rizatriptan benzoate from the solution.

### PRIORITY

This application claims the benefit under 35 U.S.C. §119 to U.S. Provisional Application No. 60/734,561, filed on Nov. 8, 2005, and entitled “AMORPHOUS RIZATRIPTAN BENZOATE” and Indian Provisional Application No. 1323/MUM/2005, filed on Oct. 20, 2005, the contents of each of which are incorporated by reference herein.

### BACKGROUND OF THE INVENTION

1. Technical Field

The present invention generally relates to an amorphous form of rizatriptan benzoate and a process for its preparation.

2. Description of the Related Art

Rizatriptan benzoate, also known as N,N-dimethyl-5-(1H-1,2,4-triazol-1-ylmethyl)-1H-indole-3-ethanamine monobenzoate, can be represented by the structure of Formula I. 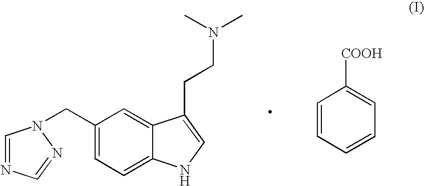
Rizatriptan binds with high affinity to human cloned 5-HT_{1B} and 5-HT_{1D} receptors. Rizatriptan has weak affinity for other 5-HT₁, receptor subtypes (5-HT_{1A}, 5-HT_{IE}, 5-HT_{IF}) and the 5-HT₇ receptor, but has no significant activity at 5-HT₂, 5-HT₃, alpha- and beta-adrenergic, dopaminergic, histaminergic, muscarinic or benzodiazepine receptors. Rizatriptan benzoate is marketed under the brand name of MAXALT® and indicated for the acute treatment of migraine attacks with or without aura in adults. See, e.g., The Merck Index, Thirteenth Edition, 2001, p. 1480, monograph 8324; and Physician's Desk Reference, “Maxalt,” 58th Edition, p. 2013-2017 (2003).

Processes for the preparation of rizatriptan benzoate are known. See, e.g., GB-A-2315673; WO-A-95/32197; EP-A-497512; Cheng-yi Chen et al., Tetrahedron Letters, Vol. 35, pp. 6981-6984 (1994) and L. J. Street et al., Journal of Medicinal Chemistry, Vol. 38, pp. 1799-1810 (1995). Each of the references disclose rizatriptan benzoate being isolated from ethanol as a white solid with a melting point of 178-180° C.

WO 2005/068453 (“the '453 application”) discloses polymorphic Forms A and B of rizatriptan benzoate. The '453 application further discloses that crystallization from a C₁-C₈ alcohol produces Form A.

The amorphous forms in a number of drugs exhibit different dissolution characteristics and in some cases different bioavailability patterns compared to crystalline forms. See, e.g., Konne T., Chem Pharm Bull, 38, 2003 (1990). For some therapeutic indications, one bioavailability pattern may be favored over another. An amorphous form of cefuroxime axietil is an example of one amorphous drug exhibiting much higher bioavailability than the crystalline forms, which leads to the selection of the amorphous form as the final drug substance for cefuroxime axietil pharmaceutical dosage form development. Additionally, the aqueous solubility of crystalline atorvastatin calcium is lower than its amorphous form, which may result in the difference in their in vivo bioavailability. An amorphous form of rizatriptan benzoate has now been discovered.

### SUMMARY OF THE INVENTION

In accordance with one embodiment of the present invention, an amorphous form of rizatriptan benzoate is provided.

In accordance with a second embodiment of the present invention, a process for preparing an amorphous form of rizatriptan benzoate is provided, the process comprising the steps of:

(a) preparing a solvent solution comprising non-amorphous rizatriptan benzoate and one or more solvents capable of dissolving the non-amorphous rizatriptan benzoate; and

(b) recovering the amorphous form of rizatriptan benzoate from the solution.

In accordance with a third embodiment of the present invention, a pharmaceutical composition is provided comprising a therapeutically effective amount of an amorphous form of rizatriptan benzoate.

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “subject” or “a patient” or “a host” as used herein refers to mammalian animals, preferably human.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, which is incorporated herein by reference in its entirety.

The term “diluent” or “filler” as used herein is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, polyvinylpyrrolidone (PVP), tyloxapol (a nonionic liquid polymer of the alkyl aryl polyether alcohol type, also known as superinone or triton), combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### BRIEF DESCRIPTION OF THE DRAWING

FIG. 1 is a characteristic powder X-ray diffraction (XRD) pattern of rizatriptan benzoate in an amorphous form.

### DETAILED DESCRIPTION OF THE PREFERRED EMBODIMENTS

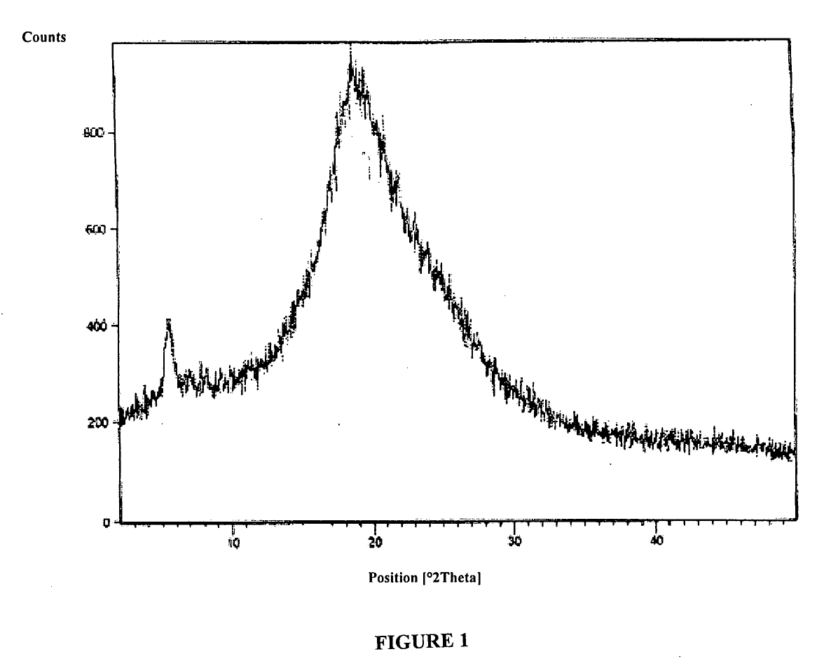

The present invention provides rizatriptan benzoate in an amorphous form. In one embodiment, the present invention provides rizatriptan benzoate substantially in an amorphous form. Substantially amorphous rizatriptan benzoate contains a very low content of crystalline rizatriptan benzoate, e.g., less than about 5% crystallinity, preferably less than about 2%, and more preferably less than about 1% crystallinity. Crystallinity may be measured using methods familiar to those skilled in the art. Exemplary methods include, but are not limited to, powder X-ray diffraction, differential scanning calorimetry, dynamic vapor sorption, isothermal microcalorimetry, inyerse gas chromatography, near infra-red spectroscopy, solid-state NMR and the like. The XRD pattern of the amorphous rizatriptan benzoate of the present invention is substantially in accordance with FIG. 1. The x-ray powder diffractogram was measured on a Xpert pro analytical diffractometer.

Generally, rizatriptan benzoate in amorphous form can be prepared by at least (a) preparing a solvent solution containing non-amorphous rizatriptan benzoate and one or more solvents capable of dissolving the non-amorphous rizatriptan benzoate; and (b) recovering the amorphous form of rizatriptan benzoate from the solution.

In step (a) of the process of the present invention, non-amorphous rizatriptan benzoate is dissolved in a solvent solution containing one or more solvents capable of dissolving rizatriptan benzoate to provide a clear solution. The non-amorphous rizatriptan benzoate used as a starting material in the process can be any known non-amorphous rizatriptan benzoate.

Suitable solvents for use herein include, but are not limited to, water, organic solvents, e.g., lower alcohols and the like, and mixtures thereof. Suitable alcohol-containing solvents include aromatic and aliphatic C₁-C₁₂ alcohols and the like and mixtures thereof. Suitable aliphatic alcohols include C₁-C₈ alcohols such as, for example, methanol, ethanol, n-propanol, isopropanol, n-butanol, isobutanol, tert-butanol and the like and mixtures thereof. Suitable aromatic alcohols include C₃-C₁₂ alcohols such as, for example, benzyl alcohol, benzyloxyethanol, phenoxyethanol and the like and mixtures thereof. Preferably the solvent is water or mixtures of water and an alcohol. Generally, the solvent can be present in an amount sufficient to dissolve the starting material, e.g., an amount ranging from about 2 to about 30% w/v and preferably from about 5 to about 10% w/v. The dissolution can be carried out at a temperature ranging from about 20° C. to about 80° C. and preferably at room temperature.

If desired, the clear solution of step (a) can be filtered to remove any extraneous matter present in the solution using any standard filtration techniques known in the art. A filtering aid such as celite can be added to the solution to assist in the filtration of the extraneous matter.

In step (b) of the process of the present invention, rizatriptan benzoate in an amorphous form is recovered from the solution. For example, amorphous rizatriptan benzoate is recovered from the solution by substantially removing the solvent from the solution to provide amorphous rizatriptan benzoate as, for example, a free-flowing powder. The solvent may be removed by techniques well known in the art, for example, substantially complete evaporation of the solvent, concentrating the solution, cooling to a temperature sufficient to precipitate an amorphous form and filtering the solid under nitrogen atmosphere. In one embodiment, amorphous rizatriptan benzoate can be recovered by spray drying the solution.

When removing the solvent by evaporation, evaporation can be achieved at sub-zero temperatures by the lyophilisation or freeze-drying technique. The solution may also be completely evaporated in a pilot plant Rota vapor, a Vacuum Paddle Dryer or in a conventional reactor under vacuum above about 720 mm Hg by flash evaporation techniques at a temperature of about 90° C., using an agitated thin film dryer (“ATFD”), or evaporated by spray drying at a temperature ranging from about room temperature to about 90° C. to obtain a dry amorphous powder. When the solvent is water, the solvent may be removed by distillation under vacuum in a pilot plant Rota vapor.

In another embodiment, the solution may be concentrated under vacuum, for example, about 720 mm Hg. For example, the initial mass is concentrated to about 1 to about 3 volumes. The concentration may also take place in, for example, a reactor with stirring, a rota vapor or vacuum paddle dryer with stirring. After concentrating the solution, the solution can then be cooled to a temperature of about 0° C. to obtain a slurry. The slurry can then be filtered under controlled conditions using standard filtration techniques such as over a Nutsche filter, Agitated Nutsche filter, centrifugation, through a filter press or in a sparkler filter. Filtration can typically be carried out under controlled conditions such as, for example, a nitrogen atmosphere, a temperature of about 25° C. and a relative humidity ranging from about 45% to about 50%. The wet product may then be dried. Drying may be accomplished by evaporation, spray drying, drying under vacuum, or freeze-drying. In one embodiment, the wet product can be dried at a temperature of about 60° C.

The substantially pure amorphous rizatriptan benzoate obtained by the above processes may be further dried in, for example, Vacuum Tray Dryer, Rotocon Vacuum Dryer, Vacuum Paddle Dryer or pilot plant Rota vapor, to further lower residual solvents.

Another embodiment of the present invention is directed to a process for producing pure amorphous rizatriptan benzoate. Generally, the process includes (a) preparing an organic solvent solution containing at least rizatriptan base, amorphous or crystalline, in an alcoholic solvent capable of dissolving the starting material; (b) adding an aqueous or organic solvent solution containing at least benzoic acid to the above solution of the base to provide a clear solution in which the homogeneity of the clear solution is maintained or a slurry containing the non-amorphous rizatriptan benzoate salt; (c) optionally filtering the clear solution to remove any extraneous matter in the case of aqueous or aqueous alcoholic solution; and (d) recovering substantially pure amorphous rizatriptan benzoate from the solution, e.g., by concentrating or evaporating the solution with or without heating solution at atmospheric pressure or under vacuum or filtration of the non-amorphous form of rizatriptan benzoate followed by dissolution of the non-amorphous form in water or lower alcoholic solvents such as methanol or ethanol or the mixtures thereof and evaporating the solvent by methods such as, for example, freeze drying or spray drying as described hereinabove. The concentration or evaporation may be carried out by any of the above described techniques.

Yet another embodiment of the present invention is directed to pharmaceutical compositions containing at least a therapeutically effective amount of the amorphous rizatriptan benzoate of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The amorphous rizatriptan benzoate of the present invention may also be administered as suppositories, ophthalmic ointments and suspensions, and parenteral suspensions, which are administered by other routes. The dosage forms may contain the amorphous rizatriptan benzoate of the present invention as is or, alternatively, as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain the amorphous rizatriptan benzoate of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

In one embodiment, the amorphous rizatriptan benzoate of the present invention for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. It is noted the notation D_{X}, means that X % of the particles have a diameter less than a specified diameter D. Thus, a D₅₀ of about 400 microns means that 50% of the micronized particles in a composition have a diameter less than about 400 microns. The term “micronization” used herein means any process or methods by which the size of the particles is reduced. For example, the particle sizes of the amorphous rizatriptan benzoate of the present invention can be obtained by any milling, grinding, micronizing or other particle size reduction method known in the art to bring the solid state forms into any of the foregoing desired particle size range.

Actual dosage levels of the amorphous rizatriptan benzoate of the present invention may be varied to obtain an amount that is effective to obtain a desired therapeutic response for a particular composition and method of administration. The selected dosage level therefore depends upon such factors as, for example, the desired therapeutic effect, the route of administration, the desired duration of treatment, and other factors. The total daily dose of the compounds of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. The pharmaceutical compositions herein can formulated in any release form, e.g., immediate release, sustained release, controlled release, etc.

The following examples are provided to enable one skilled in the art to practice the invention and are merely illustrative of the invention. The examples should not be read as limiting the scope of the invention as defined in the claims.

### EXAMPLE 1

Preparation of Non-Amorphous Rizatriptan Benzoate

Rizatriptan base (1.0 kg) amorphous was dissolved in isopropyl alcohol (8.5 L) at room temperature, a temperature ranging from about 25° C. to about 30° C. Benzoic acid (0.49 kg) was dissolved in isopropyl acetate (3.5 L). The benzoic acid solution was added slowly to the solution of the rizatriptan base under nitrogen atmosphere with stirring. The reaction mixture was stirred for about 1 hour and filtered under nitrogen atmosphere. The filtrate was washed with isopropyl acetate (1.0 L) and dried at a temperature of about 60° C. to obtain a non-amorphous rizatriptan benzoate. Weight: 1.0 kg.

### EXAMPLE 2

Preparation of Non-Amorphous Rizatriptan Benzoate

Non-amorphous rizatriptan benzoate of Example 1 (1.0 kg) was dissolved in isopropyl alcohol (8.5 L) at room temperature. Benzoic acid (0.49 kg) was dissolved in isopropyl acetate (3.5 L). The benzoic acid solution was added slowly to the solution of the rizatriptan base under nitrogen atmosphere with stirring. The reaction mixture was stirred for about 1 hour and filtered under nitrogen atmosphere. The filtrate was washed with isopropyl acetate (1.0 L), and dried at a temperature of about 60° C. to obtain a non-amorphous rizatriptan benzoate. Weight: 1.0 kg.

### EXAMPLE 3

Preparation of Amorphous Rizatriptan Benzoate

Non-amorphous rizatriptan benzoate of Example 1 (1.0 kg) was dissolved in water at room temperature and filtered through a filtration medium (or filter aid) to remove extraneous matter. The clear solution was spray-dried in a ‘Lab-plant’ model spray drier at a temperature of about 90° C. to obtain a dry free-flowing amorphous powder. Weight: 0.7 kg.

### EXAMPLE 4

Preparation of Amorphous Rizatriptan Benzoate

Non-amorphous rizatriptan benzoate of Example 1 (1.0 kg) was dissolved in water at room temperature and filtered through a filtration medium (or filter aid) to remove extraneous matter. The clear solution was subjected to distillation in a pilot plant Rota vapor under high vacuum until a dry free-flowing amorphous powder was obtained. Weight: 1.0 kg.

### EXAMPLE 5

Preparation of Amorphous Rizatriptan Benzoate

Rizatriptan base (1.0 kg) was dissolved in methanol (8.5 L) at room temperature. Benzoic acid (0.49 kg) was dissolved in methanol (5 L). The benzoic acid solution was added slowly to the solution of the rizatriptan base under nitrogen atmosphere with stirring. The reaction mixture was stirred for about 1 hour. Water (5.0 L) was added to the clear rizatriptan benzoate solution. Methanol was stripped off under vacuum completely and the resulting in the aqueous solution of the benzoate salt, which is then filtered to remove any extraneous matter. The clear solution was subjected to lyophilisation for 24 hours until a free flowing amorphous solid is obtained.

Alternatively the amorphous rizatriptan benzoate can be isolated from the filtered aqueous solution by spray drying or distillation of water under high vacuum to get a free-flowing amorphous powder. Weight: 1.0 kg.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. Rizatriptan benzoate in an amorphous form.

2. The amorphous rizatriptan benzoate of claim 1, containing less than about 5% crystalline rizatriptan benzoate.

3. A pharmaceutical composition comprising a therapeutically effective amount of the rizatriptan benzoate in an amorphous form.

4. The pharmaceutical composition of claim 3, further comprising a pharmaceutically acceptable carrier.

5. The pharmaceutical composition of claim 3, further comprising a pharmaceutically acceptable excipient.

6. The pharmaceutical composition of claim 3, which is in the form of a tablet or capsule.

7. The pharmaceutical composition of claim 3, wherein the amorphous rizatriptan benzoate is a micronized amorphous rizatriptan benzoate having a particle size of less than about 100 microns.

8. A process for preparing rizatriptan benzoate in an amorphous form, the process comprising the steps of:
(a) preparing a solvent solution comprising non-amorphous rizatriptan benzoate and one or more solvents capable of dissolving the non-amorphous rizatriptan benzoate; and
(b) recovering the amorphous form of rizatriptan benzoate from the solution.

9. The process of claim 8, wherein the solvent is selected from the group consisting of water, alcohol and mixtures thereof.

10. The process of claim 8, wherein the alcohol is selected from the group consisting of methanol, ethanol, isopropanol and mixtures thereof.

11. The process of claim 8, wherein the step of recovering comprises distilling the solvent from the solution.

12. The process of claim 8, wherein the step of recovering comprises evaporating the solvent from the solution.

13. The process of claim 8, wherein the step of recovering comprises spray drying.

14. The process of claim 8, wherein the amorphous rizatriptan benzoate recovered in step (b) is substantially pure.

15. The process of claim 8, wherein the amorphous rizatriptan benzoate recovered in step (b) has a purity greater than or equal to about 98%.

16. Rizatriptan benzoate in an amorphous form and having a purity greater than or equal to about 98%, prepared in accordance with claim 8.
